(19) United States
(12) Patent Application Publication
(10) Pub. No.: US 2007/0202058 A1
(43) Pub. Date: Aug. 30, 2007

(54) Compositions for treating gastric reflux

(75) Inventor: Gary Jim Calton, Elkridge, MD (US)

Correspondence Address: Gary Calton, 5331 Landing Road, Elkridge, MD, 21075, US

(73) Assignee: Calwood Nutritionals, Inc.

(21) Appl. No.: 11/711,307

(22) Filed: Feb. 27, 2007

Related U.S. Application Data
(60) Provisional application No. 60/777,494, filed on Feb. 27, 2006.

(51) Int. Cl. A61K 31/195; A61K 31/19; A61K 9/68

(52) U.S. Cl. 424/48; 514/557; 514/561

(57) ABSTRACT

Methods and compositions for treating gastric reflux or the pain associated therewith comprising orally administering therapeutically effective amounts of certain amino acids in a pharmaceutically acceptable composition are described herein.

### CROSS-REFERENCE TO RELATED APPLICATIONS

This application claims the benefit of Provisional Patent Application 60/777,494, filed Feb. 27, 2006, by the present inventor.

### FEDERALLY SPONSORED RESEARCH

Not Applicable

### SEQUENCE LISTING OR PROGRAM

Not Applicable

### FIELD OF THE INVENTION

The present invention relates to methods and pharmaceutical compositions for use in treating gastroesophageal irritation, nausea and pain associated with gastric reflux.

### BACKGROUND OF THE INVENTION

Esophageal pain, commonly experienced as heartburn, is symptomatic of gastric reflux. Gastric reflux occurs when small amounts of gastric juice and/or bile acids pass into the lower part of the esophagus and cause esophageal irritation. Typically, gastric reflux, which occurs after meals, especially large meals, is aggravated by bending over or lying down, and is a common occurrence in patients having a hiatal hernia, or a weakening of the esophageal sphincter. Severe episodes of gastric reflux may inflame the esophageal mucosa and lead to the more serious condition of reflux esophagitis in which severe damage or loss of squamous epithelium of the lower part of the esophagus may occur. If esophagitis is persistent or severe, an inflammatory blockage of the esophagus may develop.

Persistent gastric reflux has been treated by attempting to reduce gastric volume, acidity of the gastric contents, and accelerated gastric emptying. Reduction in gastric pH is commonly effected by frequent ingestion, for example, in hourly intervals, of antacid preparations such as aluminum hydroxide gel or a carbonate or bicarbonate salt. Other methods include the administration of drugs such as bethanechiol and metachlopramide, which increase the tone of the lower esophageal sphincter and accelerate gastric emptying. If these methods do not reverse the inflammatory process, surgical therapy is often recommended.

Another approach to the problem of gastric reflux comprises the administration of a preparation which forms a foam or raft which floats on the stomach contents. The foam containing antacid precedes the stomach contents into the esophagus when reflux occurs and helps to protect the mucosa from further irritation. The gelatinous foam is formed by the combination of an acid insoluble gelatinous material entrapping CO₂ gas. Heretofore known preparations used to create the foam comprise sodium bicarbonate and either solid compositions or liquid suspensions of alginic acid or its sodium salt. Exemplary of such prior art preparations include the product Gaviscon™ (Marion Laboratories) and compositions described in U.S. Pat. No. 4,140,760.

Such known compositions contain relatively small amounts of antacid material and relatively large amounts of sodium. Accordingly, they are not particularly effective when used by patients who require a substantial adjustment of gastric pH and/or problems can be encountered when they are used by patients who should not receive an excessive amount of sodium. Additionally, they are often ineffective providing only minor relief from nausea and burning, even when taken often (hourly for instance) over lengthy periods (days or weeks).

The symptoms of gastro-esophageal reflux can resemble those of a peptic ulcer, chest pains (angina pectoris), muscle pains, back problems, constipation, irritable bowel syndrome, gallstones, pancreatic disease, etc. These conditions must sometimes be ruled out before an accurate diagnosis can be made.

In the treatment of the peptic ulcer disease current therapy aims at reducing the gastric acid secretion, thus resulting in a recess of the injuries in the gastro-intestinal tract. Inhibitors of gastric acid secretion, proton pump inhibitors in particular, induce a relief of pain and other symptoms associated with the ulcer disease. However, relapses of the disease are a documented fact.

Compounds with histamine H₂-blocking activity may be used in the treatment of conditions where there is a hypersecretion of gastric acid, e.g. in gastric and peptic ulceration, however, the relief offered by such compounds is not immediate, but such compounds are most effective if taken before eating foods that may cause gastric acid. The relief associated may occur within 15-30 minutes of the patient taking an acid blocking treatment and thus may also require an antacid to relieve pain and discomfort until such time as the blocking of acid secretion takes place as for example Pepcid AC™ and Pepcid Complete™, containing the active ingredient famotidine and in the case of Pepcid Complete™, an antacid, calcium carbonate (products of Merck & Co.). Immediate relief is desirable and calcium carbonate often fails to provide relief, even when combined with famotidine.

Proton pump inhibitors such as omeprazole, and its related family of inhibitors, are used to treat severe gastric reflux, erosive esophagitis and duodenal and gastric ulcers as well as the hypersecretory disorders. When an episode of gastric reflux occurs, usually when the patient lays down, taking this class of drugs will effectively relieve the pain after 15-30 minutes. Immediate relief is desirable.

U.S. Pat. No. 3,988,466 reports that amino acids, particularly, L-glutamine are effective for the prevention or treatment of certain experimental ulcers, induced artificially by stress or pylorus ligation, or chemically by histamine, reserpine, cortisone, or inflammatory agents when given simultaneously. Further, such prevention only occurred when the concomitant dosage of the amino acid, L-glutamine, present was 2-5 g for a dose of aspirin of 0.3-1.0 g or for 25-50 mg of indomethacin, the required dose of L-glutamine was 2 g. Glutamine was completely ineffective in prevention of aspirin induced gastric lesions at a dose of 62.5 mg/kg (Table 1) which is equivalent to 4.375 g for an human adult (70 kg weight). Glutamine was ineffective in prevention of indomethacin induced gastric lesions at a dose of 15.6 mg/kg (Table 2) which is equivalent to 1.1 g for an human adult (70 kg weight). No use or indication for relief of gastric distress before or after lesion formation was observed, nor could it have been in the animal model used. No use or indication for relief of gastric lesions after lesion formation was disclosed. The lower level of use claimed (1 to 10 grams, claim 1) is shown by the specification to be ineffective as the minimum effective dose was 125 mg/kg (Table 1) to 31.3 mg/kg (Table 2) or 8.75 g and 2.2 g for a 70 kg human. Of the 24 amino acids tried at the pharmaceutically unacceptable level of 750 mg/kg (calculated to be 52.5 g of the amino acid for a 70 kg human dose), all but DL-tryptophan, L-aspartic acid, L-tyrosine, L-cysteine and L-cystine gave more than 50% inhibition of ulcers. The pH of the amino acids was not a factor in ulcer prevention, showing that “the effect of amino acids is different from that of antacids” (Col 6, lines 27-31)

Sukumar et. al. found that glutamine in a guar gum had no effect on ulcer healing, and that L-arginine, although somewhat effective in ulcer healing, was antagonistic to ulcer healing by yeast RNA (56 vs 34% decrease in ulcer number) in rats (Sukumar P, Loo A, Magur E, Nandi. J, Oler A, Levine R A. Dietary supplementation of nucleotides and arginine promotes healing of small bowel ulcers in experimental ulcerative ileitis. Dig Dis Sci. 1997 July; 42(7):1530-6.).

A double blind trial found that arginine may increase the risk of esophageal reflux (heartburn) by relaxing the sphincter at the bottom of the esophagus. (Luiking Y C, Weusten B L, Portincasa P, et al. Effects of long-term oral L-arginine on esophageal motility and gallbladder dynamics in healthy humans. Am J. Physiol. 1998; 274)

### SUMMARY OF THE INVENTION

The present invention relates to a method for the treatment of gastric reflux, heartburn and nausea, comprising an effective amount of a nitric oxide releasing compound or composition.

The present invention relates to a method for the treatment of gastric reflux and nausea, comprising an effective amount of an amino acid selected from the group of amino acids consisting of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof in a pharmaceutically acceptable composition.

A further aspect of the present invention relates to compositions for the administration of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof for the treatment of gastrointestinal distress.

Yet another aspect of the present invention relates to compositions for the administration of slowly released L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof for the treatment of gastrointestinal distress.

Still another aspect of the present invention relates to compositions useful in the treatment of gastric reflux and nausea, prepared from a gum and an effective amount of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof.

A method of relieving the pain associated with irritation, diseases and/or lesions in the mouth, esophagus, throat, or pharynx caused by or associated with gastric reflux, said method comprising orally administering in a suitable formulation, L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof or their physiologically acceptable salts, in an amount sufficient to reduce said irritation, diseases and/or lesions in the mouth, esophagus, throat, or pharynx. A preferred dosage is in the range of 0.1 to 15 g per episode and a more preferred dosage is in the range of 0.1 to 5 g per episode and even more preferable is a dosage in the range of 0.1 to 2.8 g per episode.

A method of treating or preventing pain or irritation, caused by or associated with gastric reflux, which method comprises administering to a patient in need of such treatment a pharmaceutically effective amount of a composition comprising from 0.1 to 99.9 percent by weight of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof. A preferred composition comprises 0.1 to 50 percent by weight of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof.

A method of treating or preventing pain caused by or associated with gastric reflux, comprising administering to a patient in need of such treatment a pharmaceutically effective amount of a composition comprising
(a) from 0.1 to 99.9 percent by weight of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof; and,
(b) from 0.1 to 50.0 percent by weight of a gum selected from alginate, locust bean gum, xanthan gum, carrageenan, konjac mannan and mixtures thereof.

A method of treating or preventing pain comprising administering to a patient in need of such treatment a pharmaceutically effective amount of a composition comprising:
(a) from 0.1 to 99.9 percent by weight of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof;
(b) from 0.1 to 50.0 percent by weight of a gum selected from alginate, locust bean gum, xanthan gum, carrageenan, konjac mannan and mixtures thereof; and
(c) from 0.1 to 50 percent by weight of an acid.
   A preferred composition contains (a) in the amount of 5 to 50 percent by weight and component (b) in the amount of from 0.1 to 50 percent by weight. The alginate may be present as a sodium, potassium or ammonium salt and sodium alginate is especially preferred.

One useful composition of the invention comprises water, 10-50%; organic acid, 1-30%; L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof, 1-50%; sweetener, 0.01-30%; flavor, as required; vegetable oil, 0.1-10%; and carrageenan, 1-50%.

Another useful composition comprises: water, 0-95%; an organic acid or phosphoric acid, 1-30%; L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof, 1-50%; sweetener, 0.01-30%; and flavor, as required.

Especially useful organic acids are citric acid, malic acid, aspartic, lactic, and fumaric acid or mixtures thereof.

Useful additions to the compositions of the invention include pharmaceutical compositions which cause gastrointestinal upset, or medicaments which relieve gastric distress.

Medicaments known to cause gastric distress which may be taken with or included in the compositions of the invention include one or more pharmaceutically active ingredients selected from the group of analgesics consisting of: celocoxib, valdecoxib, rofecoxib, acetaminophen; ibuprofen; naproxen; diclofenac; meloxicam; nabumetone; ketoprofen; etodolac; sulindac; indomethacin; oxaprozin; piroxicam; ketorolac; choline salicylate; benzydamine; buprenorphine; hydrocortisone; betamethasone; and pharmaceutically acceptable mixtures thereof.

Medicaments known to cause gastric distress which may be taken with or included in the compositions of the invention include a pharmaceutically active ingredient selected from the group consisting of: decongestants such as pseudoephedrine, phenylephrine, oxymetazoline and xylometazoline; cough suppressants such as dextromethorphan, codeine and pholocodine; expectorants such as guaiphenesin, N-acetylcysteine, and bromhexine; and pharmaceutically acceptable mixtures thereof.

Medicaments known to cause gastric distress which may be taken with or included in the compositions of the invention include a pharmaceutically active ingredient selected from the group consisting of: antiseptics such as triclosan, chloroxylenol, amylmetacresol, hexylresorcinols, dichlorobenzyl alcohol and benzyl alcohol; and pharmaceutically acceptable mixtures thereof.

Medicaments known to cause gastric distress which may be taken with or included in the compositions of the invention include a pharmaceutically active ingredient selected from the group consisting of: cardiovascular agents such as glyceryl trinitrate; and pharmaceutically acceptable mixtures thereof.

Medicaments known to cause gastric distress which may be taken with or included in the compositions of the invention include a pharmaceutically active ingredient selected from the group consisting of: local anaesthetics such as benzocaine and lignocaine; and pharmaceutically acceptable mixtures thereof.

Medicaments known to relieve gastric distress which may be taken with or included in the compositions of the invention include a pharmaceutically active ingredient selected from the group consisting of: antacid agents such as calcium carbonate, sodium bicarbonate, magnesium trisilicate, aluminum hydroxide and magaldrate; and pharmaceutically acceptable mixtures thereof.

Medicaments known to relieve gastric distress which may be taken with or included in the compositions of the invention include a pharmaceutically active ingredient selected from the group consisting of: antiulcer agents such as carbenoxolone, sucralfate, cimetidine, ranitidine, nizatidine, famotidine, omeprazole, lansoprazole, esomeprazole, raberprazole and pantoprazole; and pharmaceutically acceptable mixtures thereof.

Medicaments known to cause gastric distress which may be taken with or included in the compositions of the invention include a pharmaceutically active ingredient selected from the group consisting of: antihistamines such as loratidine, terfenadine, diphenhydramine, chlorphenhydramine, triprolidine and acrivastine; and pharmaceutically acceptable mixtures thereof.

Medicaments known to cause gastric distress which may be taken with or included in the compositions of the invention include a pharmaceutically active ingredient selected from the group consisting of: antinausea agents such as prochlorperazine and sumatriptan; and pharmaceutically acceptable mixtures thereof.

Medicaments known to cause gastric distress which may be taken with or included in the compositions of the invention include a pharmaceutically active ingredient selected from those for bowel regulation exemplified by diphenoxylate, loperamide, and sennosides; and pharmaceutically acceptable mixtures thereof.

Medicaments known to cause gastric distress which may be taken with or included in the compositions of the invention include a pharmaceutically active ingredient selected from those for antifungal agents exemplified by clotrimazole; and pharmaceutically acceptable mixtures thereof.

Medicaments known to cause gastric distress which may be taken with or included in the compositions of the invention include a pharmaceutically active ingredient selected from those for antimicrobial activity exemplified by fusafungine and tyrothricine; and pharmaceutically acceptable mixtures thereof.

The present invention includes methods of treating irritation and pain in the mouth, esophagus, throat or pharynx caused by or associated with gastric reflux, said method comprising orally administering in a suitable formulation, a composition or compound capable of releasing nitric oxide in the oropharyngeal cavity, in an amount sufficient to reduce said irritation, diseases and/or lesions in the mouth, esophagus, throat, or pharynx.

### DETAILED DESCRIPTION

### Example 1 Konjac-Arginine Confection

To 10 g water containing 0.03 g sucralose and 0.03 g lemon extract solution (McCormick) is added with vigorous stirring 1.0 g konjac mannan flour. The stirred mix becomes a stiff gel within 2 minutes (slight exotherm). To this gel is added a homogeneous blend of 3.5 g L-lysine and 1.9 g citric acid. Vigorous stirring for several minutes converts the initial stiff mush of these ingredients to a thick fluid homogeneous slurry. This slurry is cast out into a ¼″ thick sheet (conveniently between polyethylene films). After standing several hours at room temperature the slurry becomes a stiff gel having a pleasant lemonade taste and good texture in the mouth.

### Example 2 Carrageenan Confection

Liquid ingredients are mixed in a suitable mixer and the acids are added. Once the acids are dissolved, L-lysine is added and the whole is stirred until homogenous. Finally, the carrageenan is added, mixed thoroughly and then the paste is poured onto a table, spread evenly, allowed to set, and cut into suitable size doses.

| | | g/lozenge |
|---|---|---|
| Water | | 1.96 |
| Citric acid | | 0.98 |
| Malic acid | | 0.78 |
| L-lysine | | 2.81 |
| Glycerol | [symbol] | 1.78 |

| | |
|---|---|
| Sucralose (25% Solution) | 0.11 |
| Flavor | 0.50 |
| Soybean Oil | 0.42 |
| Carrageenan | 2.81 |

### Example 3 Carrageenan Confection

The following is prepared in accordance with the instructions in Example 2.

| | g/lozenge |
|---|---|
| Water | 0.45 |
| Glycerol | 0.30 |
| Citric acid | 0.35 |
| L-glutamine | 1.05 |
| Sucralose (25% Solution) | 0.025 |
| Flavor | 0.01 |
| Soybean Oil | 0.1 |
| Carrageenan | 0.7 |

### Example 4

The following is prepared in accordance with the instructions in Example 2.

| | g/lozenge |
|---|---|
| Water | 0.45 |
| Glycerol | 0.30 |
| Citric acid | 0.25 |
| Malic acid | 0.2 |
| L-lysine | 1.05 |
| Sucralose (25% Solution) | 0.025 |
| Flavor | 0.125 |
| Soybean Oil | 0.1 |
| Carrageenan | 0.7 |

### Example 5 Carrageenan Confection

The following is prepared in accordance with the instructions in Example 2.

| | g/lozenge |
|---|---|
| Water | 0.45 |
| Glycerol | 0.05 |
| Citric acid | 0.25 |
| Malic acid | 0.2 |
| L-lysine | 1.05 |
| Sucralose (25% Solution) | 0.025 |
| Flavor | 0.125 |
| Soybean Oil | 0.1 |
| Carrageenan | 0.7 |

### Example 6 Carrageenan Confection

The following is prepared in accordance with the instructions in Example 2.

| | g/lozenge |
|---|---|
| Water | 0.45 |
| Citric acid | 0.25 |
| Malic acid | 0.2 |
| L-lysine | 1.05 |
| Sucralose (25% Solution) | 0.025 |
| Flavor | 0.125 |
| Soybean Oil | 0.1 |
| Carrageenan | 0.7 |

### Example 7 Drink Mix

The following powder is mixed well and then placed into 8 ounces of water, providing a pleasant tasting drink.

| | g/serving |
|---|---|
| Citric acid | 1.68 |
| Malic acid | 0.17 |
| L-lysine | 2.8 |
| Sucralose | 0.04 |
| Flavor | 0.01 |

### Example 8

A patient having ulcerations in the upper small intestine who routinely took omeprazole or lansoprazole complained of heartburn on laying down. On successive days, the patient took the compositions of Examples 1-7. In each case the heartburn was alleviated. The patient ceased taking omeprazole or lansoprazole and instead took one of the compositions of Examples 1-7 to control gastric reflux. Immediately upon experiencing gastrointestinal distress, the patient took a dose of one of the L-lysine compositions and experienced immediate relief (usually within 10 seconds, completely subsiding within 2 minutes).

### Example 9

A patient suffering from gastric reflux due to stress for whom calcium carbonate antacids were ineffective and who was taking Nexium (esomeprazole), 40 mg twice a day, with continued heartburn, took a 1 gram dose of L-lysine as a carrageenan confection and experienced immediate relief with complete cessation of all symptoms within one minute.

### Example 10 Carrageenan Confection with L-Glutamine

The following is prepared in accordance with the instructions in Example 2.

| | g/lozenge |
|---|---|
| Water | 0.575 |
| Citric acid | 0.125 |
| L-glutamine | 1.05 |
| Sucralose (25% Solution) | 0.025 |
| Flavor | 0.125 |
| Soybean Oil | 0.1 |
| Carrageenan | 0.7 |

The L-glutamine formulation was compared by two persons to an L-arginine composition and found to be effective at relieving heartburn but to a lower degree than the L-arginine composition.

The compositions of the present invention may also include one or more of a coloring, sweetening or flavoring agent.

Tablet compositions according to the present invention may include binding agents and other ingredients known in the art to facilitate mixing, compressing, improved palatability and long term stability of the tablet.

All publications, including but not limited to patents and patent applications, cited in this specification are herein incorporated by reference as if each individual publication were specifically and individually indicated to be incorporated by reference herein as though fully set forth herein.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A method of treating the irritation, diseases and/or lesions in the mouth, esophagus, throat, pharynx or stomach caused by or associated with gastric reflux, said method comprising orally administering in a suitable formulation an amino acid or mixture thereof selected from the group of amino acids consisting of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or their physiologically acceptable salts, in an amount sufficient to reduce said irritation, diseases and/or lesions in the mouth, esophagus, throat, pharynx or stomach.

2. The method of claim 1 wherein said amino acid is L-lysine.

3. The method of claim 1 wherein said amino acid is administered at a dosage in the range of 0.1 to 15 g per episode.

4. The method of claim 1 wherein said amino acid is administered at a dosage in the range of 0.1 to 5 g per episode.

5. The method of claim 1 wherein said amino acid is administered at a dosage in the range of 0.1 to 2.8 g per episode.

6. A method of treating or preventing diseases and/or lesions in the mouth, esophagus, throat, pharynx or stomach caused by or associated with gastric reflux, which method comprises administering to a patient in need of such treatment a pharmaceutically effective amount of a composition comprising from 0.1 to 99.9 percent by weight of an amino acid or amino acids selected from the group of amino acids consisting of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof or their physiologically acceptable salts.

7. The method of claim 6 comprising: from 0.1 to 50 percent by weight of said amino acid,

8. A method of treating diseases and/or lesions in the mouth, esophagus, throat, pharynx or stomach caused by or associated with gastric reflux, which method comprises administering to a patient in need of such treatment a pharmaceutically effective amount of a composition comprising
(a) from 0.1 to 99.9 percent by weight of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof; and,
(b) from 0.1 to 50.0 percent by weight of a gum selected from alginate, locust bean gum, xanthan gum, carrageenan, konjac mannan and mixtures thereof.

9. A method of treating diseases and/or lesions in the mouth, esophagus, throat, pharynx or stomach caused by or associated with gastric reflux, which method comprises administering to a patient in need of such treatment a pharmaceutically effective amount of a composition comprising:
(a) from 0.1 to 99.9 percent by weight of L-lysine, L-histidine, L-serine, L-valine, L-threonine, glycine, L-alanine, L-glutamine, D-glutamine, L-leucine, L-isoleucine, L-glutamic acid, L-asparagine, L-proline, L-hydroxyproline, L-methionine, L-phenylalanine, DL-tryptophan, or mixtures thereof;
(b) from 0.1 to 50.0 percent by weight of a gum selected from alginate, locust bean gum, xanthan gum, carrageenan, konjac mannan and mixtures thereof; and
(c) from 0.1 to 50 percent by weight of an acid.

10. A method according to claim 9 in which component (a) is present in the amount of 5 to 40 percent by weight and component (b) is present in the amount of from 0.1 to 30 percent by weight.

11. A method according to claim 9 in which component (b) consists essentially of carrageenan.

12. A method according to claim 9 in which the composition comprises: Water, 10-50%; Organic acid, 1-30%; L-lysine, 1-50%; sweetener, 0.01-30%; Flavor, as required; Soybean Oil, 0.1-10%; Carrageenan, 1-50%.

13. The composition of claim 9, wherein said acid is selected from among the group of organic acids consisting of citric acid, malic acid, aspartic, lactic, and fumaric acid or mixtures thereof.

14. A method of relieving the pain associated with irritation, diseases and/or lesions in the mouth, esophagus, throat, pharynx or stomach caused by or associated with gastric reflux, said method comprising:
a) said composition or compound of claim 9, and
b) a pharmaceutical composition which causes gastrointestinal upset.

15. A method of relieving the pain associated with irritation, diseases and/or lesions in the mouth, esophagus, throat, pharynx or stomach caused by or associated with gastric reflux, said method comprising:
a) said composition or compound of claim 9, and
b) a pharmaceutical composition which prevents gastrointestinal upset.
